(19) United States
(12) Patent Application Publication
(10) Pub. No.: US 2022/0218513 A1
(43) Pub. Date: Jul. 14, 2022

(54) INSTRINSICALLY LUBRICATING DRUG-LOADED HYDROGELS FOR USE AS PROPHYLACTIC MEDICAL DEVICES

(71) Applicant: THE TRUSTEES OF THE UNIVERSITY OF PENNSYLVANIA, Philadelphia, PA (US)

(72) Inventors: Robert W. CARPICK, Philadelphia, PA (US); Shu YANG, Blue Bell, PA (US); José A. BAUERMEISTER, Philadelphia, PA (US); Megan B. ELINSKI, Philadelphia, PA (US); Alexander I. BENNETT, Philadelphia, PA (US); Haihuan WANG, Philadelphia, PA (US); Wei-Liang CHEN, Newtown, PA (US); Christian POHLMANN, Fairfax Station, VA (US); Willey Y LIN, Philadelphia, PA (US)

(21) Appl. No.: 17/610,334

(22) PCT Filed: May 14, 2020
(86) PCT No.: PCT/US2020/032808

§ 371 Date: Nov. 10, 2021

Related U.S. Application Data
(60) Provisional application No. 62/847,476, filed on May 14, 2019.

(51) Int. Cl. A61F 6/04; A61L 31/14; A61L 31/16; A61L 31/10; C08L 33/26; C09D 133/26; C08L 51/04; C08L 51/08

(52) U.S. Cl. CPC A61F 6/04; A61L 31/145; A61L 31/16; A61L 31/10; C08L 33/26; C08L 2205/03; C08L 51/04; C08L 51/085; C08L 2203/02; C08L 2312/00; C09D 133/26

(57) ABSTRACT

The invention concerns personal wellness products comprising: a self-lubricating, tough hydrogel material, the hydrogel material optionally comprising a double interpenetrating network (D-IPN) matrix.

### CROSS REFERENCE TO RELATED APPLICATIONS

This invention claims benefit to U.S. Patent Application No. 62/847,476 which was filed on May 14, 2019, the disclosure of which is incorporated herein in its entirety.

### GOVERNMENT RIGHTS

This invention was made with government support under A1045008 awarded by the National Institutes of Health and under CMMI-1401164 awarded by the National Science Foundation. The government has certain rights in this invention.

### TECHNICAL FIELD

The invention concerns intrinsically lubricating drug-loaded hydrogels for use as prophylactic medical devices.

### BACKGROUND

Condoms, when properly used, are highly efficacious in reducing the spread of sexually transmitted diseases (STDs) and unintended pregnancies, having beneficial impact related to health and family planning across the developed and developing worlds. Adherence to their usage is often limited by the diminished pleasure resulting from reduced skin-to-skin contact, due to different frictional sensation and reduced thermal transfer. Moreover, natural rubber latex, the principal material used for condom manufacturing, does suffer from limits including high friction that can lead to discomfort and mucosal tissue damage, allergic reactions, and slippage or breakage; one recent study reported over one third of sexually active condom users experiencing condom failure within the past 6 months.

Products used during receptive anal intercourse (RAI) (e.g., condoms) typically require external lubricants which fail to provide sufficient lubrication, leading to their inconsistent use, increased rectal trauma during RAI, and heightened biologic vulnerability to HIV and sexually transmitted infections (STIs). There is a need for an improved product.

### SUMMARY

Hydrogels can comprise a double network matrix, a chemical network and an ionic network. The chemical network provides mechanical strength due to covalent bonding while the ionic network facilitates energy dissipation leading to high toughness while preserving extreme elasticity. Internetwork connections between the polymers preserve properties from both networks.

In some embodiments, the invention concerns personal wellness products comprising: a self-lubricating, tough hydrogel material, the hydrogel material optionally comprising a double interpenetrating network (D-IPN) matrix. In certain embodiments, the personal healthy product being characterized as a condom, a sexual health device, or a sexual pleasure device. In certain preferred embodiments, the personal wellness product is substantially free of any additional external lubricant.

Some personal wellness products comprise hydrogel material disposed as a coating on a base material. Certain personal wellness products have a base material comprising a polymer network, preferably elastomers, such as latex, polyurethane and silicone. Other personal wellness products have the hydrogel material as a free-standing without a base material.

In certain preferred embodiments, the hydrogel material comprises one or more medicaments. Medicaments include, but are not limited to, Tenofovir/tenofovir disoproxil fumarate, Emtricitabine, Dapivirine, Maraviroc, Vicriviroc, MK-2048/2048A, Levonorgestrel (birth control), MIV-150, UC781, Alafenamide, and Elvitegravir.

In some embodiments, the hydrogel material comprises one or more antifouling products.

Certain hydrogel materials comprise one or more of biocompatible and bioactive polymers such as chitosan, hyaluronic acid (HA), alginate, polyacrylamide (PAm), poly(acrylic acid) (PAA), poly(methacrylic acid) (PMAA), poly(vinyl alcohol) (PVA), and poly({2-[methacryloyloxy]ethyl}trimethylammonium chloride) (PMETAC). Importantly, the properties of the hydrogels can be tuned over physiologically-necessary ranges by varying the network's crosslinking density and composition. Their Young's moduli can range from a few kPa to a few MPa, comparable to different cell types and other soft tissues. D-IPN can be prepared from alginate-PAm, PAA-poly(ethylene oxide) (PEO), HA-poly(N,N′-dimethylacrylamide) (PDMA), poly (2-acrylamido, 2-methyl, 1-propanesulfonic acid) (PUMPS)-PAm. The minor network comprises of abundantly cross-linked polyelectrolytes (or ionic gels), providing rigid skeleton, and the major network comprises of poorly cross-linked neutral hydrophilic polymers, providing the ductile rubber network. For example, D-IPN of alginate-PAm can be crosslinked by calcium sulfate or calcium chloride.

Some preferred hydrogels comprise from 75-90 wt % water and from 10-25 wt % combined of acrylamide and alginate.

A low friction coefficient is beneficial for the instant hydrogels. In some embodiments, the product is characterized as having a friction or traction coefficient in the range of from about 1 or less. In some embodiments, the friction or traction coefficient is between 0.1-1, then between 0.01-1, then 0.001-1.

In yet other embodiments, the invention concerns a medical device comprising hydrogels disclosed herein. Medical devices include contact lenses, hygiene products, tissue engineering scaffolds, drug delivery carriers (e.g. in transdermal and ocular therapeutics, gastric retentive devices), wound dressings, needles, catheters, cannulas, trocars, endotracheal tubes, endoscopes (arthroscopes, bronchoscopes, colonoscopes, ureteroscopes, etc.), cutting edges, valves, and stopcocks. In many of these applications, low friction between the device and the tissue during sliding contact is crucial to avoid injury, pain, and discomfort.

In yet another aspect, the invention concerns methods of forming a medical product comprising the hydrogels described herein, the methods comprising:
treating a primer layer so as to form one or more reactive acrylate functional groups or initiator configured to serve as anchoring points for a hydrogel material and
anchoring the hydrogel material to the primer layer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents chemical structure and stretchability for synthesized double network hydrogel. (a) The chemicals used for hydrogel synthesis. Both chemical/ionic network components are labelled. A redox initiator pair was applied for polymerization. UV light was cast on during the polymerization for the formation of internetwork connection. (b) schematic expression for synthesized hydrogel. The internetwork connections between two networks allows forces to transmit between the two networks to attain comprehensively good mechanical properties. (c) Stretching test for the synthesized bulk hydrogel showed strain more than 20 times of its original length could be achieved without breakage or damage.

FIG. 2 shows procedure for coating method 1 (CM1) and the resulting hydrogel-coated, latex substrates. (a-b) immersing the substrate in benzophenone solution, and then the first UV exposure leads to the interface to be functionalized with benzophenone. Double network hydrogel with the same structure as shown in FIG. 1 was used as a coating solution used in step (c). After the second UV exposure, hydrogel coating is formed on the substrate as shown in step (d). A representative sample of a hydrogel-coated condom (e) and hydrogel-coated latex sheet (f) demonstrate a uniform, nearly imperceptible surface coating.

FIG. 3 shows procedure for coating method 2 (CM2). (a) generate radicals through diazonium chemistry. (b) add acrylic acid (AA) monomers into the solution and a thin layer of PAA will be formed and chemically bind on the surface. (c) graft glycidyl methacrylate onto the PAA for subsequent grafting and polymerization.

FIG. 4 shows macroscale friction measurements of hydrogel coatings in a water bath, showing the friction coefficient over total elapsed testing time. Sample, temperature, load, and sliding speed varied as specified. For tests labeled as Stribeck, the sliding speed was altered in 1 minute increments at: 10, 50, 100, 150, and 200 mm/s.

FIG. 5 presents macroscale friction measurements for commercial personal lubricants in a latex-on-latex contact.

FIG. 6 presents friction measurements on uncoated latex condoms, and gel-coated latex (bulk and condom) samples. (a) shows representative friction loops where the gel-coated condom and gel-coated latex samples have lower friction than latex condom samples with silicone based lubricant. Black squares signify the analyzed portion of the friction test. This section of the test corresponds to the sliding regime governed by kinetic friction. (b) shows the friction coefficient for these samples over 10 cycles, again the gel-coated samples perform on par or better than the latex condom sample with the best lubricant (silicone-based lubricant).

FIG. 7 presents friction coefficient vs. sliding speed for gel-coated and uncoated samples (a). (b) shows the friction coefficient dependence on UV curing light wavelength.

FIG. 8 presents a drug release curve wherein tenofovir, a pre-exposure prophylactic, is doped within the hydrogel coating prior to curing and then placed into a bath. Drug release is measured through UV-VIS spectrometry and shows a burst release of drug (up to 50% of the total drug within the hydrogel) over the course of −25 minutes.

### DETAILED DESCRIPTION OF ILLUSTRATIVE EMBODIMENTS

Condoms, when properly used, are highly efficacious in reducing the spread of sexually transmitted diseases (STDs) and unintended pregnancies, having beneficial impact related to health and family planning across the developed and developing worlds. Adherence to their usage is often limited by the diminished pleasure resulting from reduced skin-to-skin contact, due to different frictional sensation and reduced thermal transfer. Moreover, natural rubber latex, the principal material used for condom manufacturing, does suffer from limits including high friction that can lead to discomfort and mucosal tissue damage, allergic reactions, and slippage or breakage; one recent study reported over one third of sexually active condom users experiencing condom failure within the past 6 months.

In addition, products used during receptive anal intercourse (RAI) (e.g., condoms) typically require external lubricants which fail to provide sufficient lubrication, leading to their inconsistent use, increased rectal trauma during RAI, and heightened biologic vulnerability to HIV and sexually transmitted infections (STIs). Hydrogels are biocompatible materials that are intrinsically lubricious and capable of drug delivery. Despite existing studies to create tough hydrogels and claiming their potential uses in condoms, none has carefully characterized or taught us of the lubrication properties under frictional sliding, especially under different shearing speeds in vitro or in vivo, as a function of chemical composition and molecular structures of the hydrogels. The work we seek to patent is for the synthesis and characterization of novel hydrogels for condoms and other medical devices that achieve low friction and high durability without any additional lubricant, as well as incorporating prophylactic properties. The research will lead to greater usage, adherence, and reliability of sexual health products for HIV and STI prevention. The work will also have an impact for improving the surface lubricity of other polymer-based medical devices.

In some embodiments, hydrogels used with the invention can comprise a double network matrix. Such a network has both a chemical polymer network and an ionic network. The chemical network provides mechanical strength due to covalent bonding while the ionic network facilitates energy dissipation leading to high toughness while preserving extreme elasticity. Internetwork connections between the two networks preserve properties from both networks. In certain embodiments, carboxylic groups in the alginate network provide a vehicle for drug loading by forming weak hydrogen bonds between drug molecules and the hydrogel.

In some embodiments, the crosslinking interactions of the double network matrix can be composed either two of the following: covalent bonds, ionic bonds, hydrogen bonds, π-π interactions, crystallization, and hydrophobic interactions.

An example of a double network matrix is a Polyacrylamide-Alginate double network hydrogel made of both a chemical polymer network and an ionic polymer network. The chemical network provides mechanical strength due to covalent bonding while the ionic network facilitates energy dissipation leading to high toughness while preserving extreme elasticity. Internetwork connections between the polymers preserve properties from both networks. Carboxylic groups in alginate provide a vehicle for drug loading by forming weak hydrogen bonds between drug molecules and the hydrogel.

Synthesis of Bulk and Thin Coating Hydrogels:

Hydrogels are water-containing (30-99%) polymer networks whose physical properties can be fine-tuned to match biological systems. Moreover, hydrogels can be applied as thin coatings onto devices to produce low friction. Due to crosslinking and entanglements, the network forms a mesh with a size scale (ξ) on the order of 10's of nm. Hydrogel's lubricating properties can be broadly tuned over physiologically-necessary ranges by varying the network's crosslinking density and composition. Their Young's moduli can range from a few kPa to a few MPa, comparable to different cell types and other soft tissues. Polymers of interests for hydrogels include polyacrylamide (PAm), poly(acrylic acid) (PAA), poly(methacrylic acid) (PMAA), poly(vinyl alcohol) (PVA), and poly({2-[methacryloyloxy]ethyl}trimethylammonium chloride) (PMETAC). Additional components could be combined into the synthesized hydrogel to form additional networks beyond the primary covalent network in order to tune the desired mechanical properties while preserving other properties.

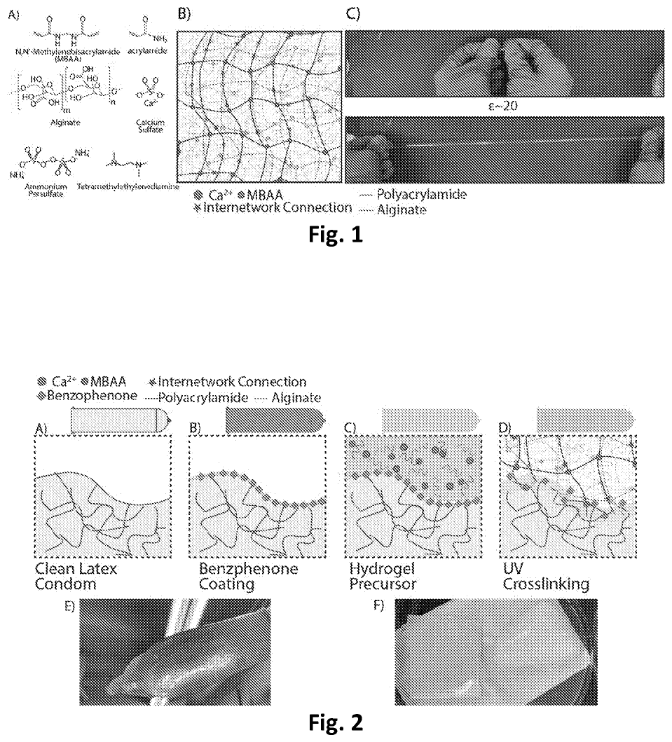

In the example here, bulk (free-standing) hydrogels have been synthesized by PAm and alginate to form double network hydrogel as shown in FIG. 1. For the hydrogel composition, the weight of the water is 75-90%, while acrylamide and alginate together are 25-10%. The ratio of acrylamide/(alginate+acrylamide) is 75-90%. For the crosslinker, MBAA is 0.03-0.12% of acrylamide and calcium sulfate is 5-50% of alginate. Ammonium persulfate is 0.1-0.01% while N,N,N′,N′-tetramethylethylenediamine is 0.1-0.01%. Generally, for the mono-component hydrogel (e.g. PAm), the friction coefficient is inversely related to the Young's modulus (controlled by the mesh size) so that a highly lubricous hydrogel has lower mechanical integrity. Through the addition of alginate and calcium, sodium, or lithium as an ionic network to chelate the alginate, the mechanical durability is improved while maintaining low friction. Moreover, FIG. 1c demonstrates that such a combination achieves extreme stretchability (λ>20), demonstrating how it is a promising candidate as a coating material for flexible/stretchable devices.

For coating the hydrogel on the substrates of supporting materials, two methods have been demonstrated that provide strong adhesion at the interface between the hydrogel and supporting material (coating method 1, CM1; coating method 2, CM2). FIG. 2 shows an illustration of CM1. Here, the hydrophobic photoinitiator benzophenone was initially pre-diffused into the polymer by immersing the substrates into a 10 wt % ethanolic initiator solution. After immersion, the substrates were treated with UV exposure. Then the substrates were cleaned with isopropyl and dried by air blowing. Subsequent exposure to ultraviolet (UV) light drove the formation of covalent bonds and entanglements between the hydrogel and the polymer surfaces. Successful coating has been demonstrated on a latex condom, a latex sheet, and a polydimethylsiloxane (PDMS) substrate. While CM1 is successful for achieving strong hydrogel grafting on polymer-based surface, it can be limited by the required diffusion of the photoinitiator into the surface.

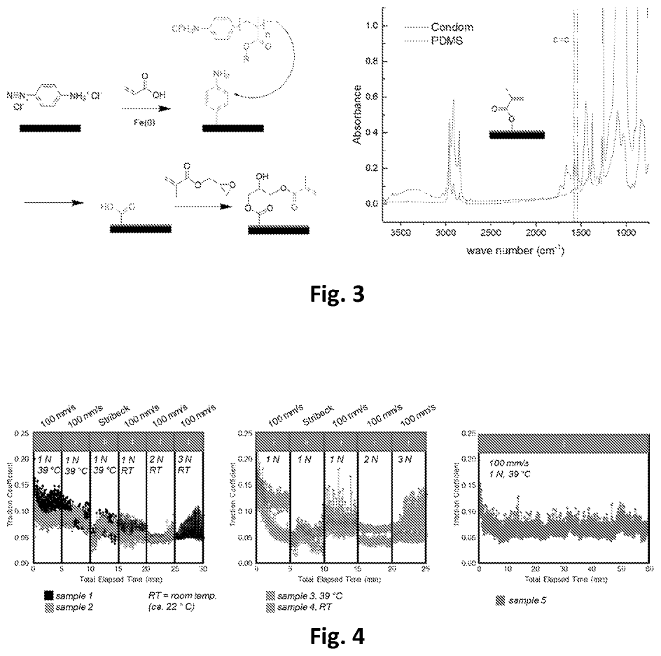

CM2 was developed to overcome the limitations of CM1 and broaden the applicability of hydrogel coatings. An overview of the procedure and example results of CM2 are shown in FIG. 3. In the first step, the in situ synthesized diazonium moieties from p-phenylenediamine and nitric acid generate radicals upon the addition of the reducing agent, Fe(0). The radical then either binds to the surface directly or induces polymerization in the solution with the added monomer, acrylic acid (AA), before surface attachment. Since radicals generated through diazonium chemistry form strong bonds on the surfaces of metal/glass/Teflon™/carbon nanotubes, by using radicals from diazonium and the AA monomer, a tightly surface-bound PAA hydrogel is provided for grafting of glycidyl methacrylate. In FIG. 3b, the FTIR spectra shows the generation of the methacrylate group on the surface (e.g., the C═C peaks at 1541 cm⁻¹ and 1577 cm⁻¹). After the formation of this methacrylate base layer on the substrate, hydrogels are then polymerized directly onto the surface via the pathway outlined as the final step in CM1, again producing strong hydrogel-substrate interfacial attachment. FIG. 3c demonstrates that the interfacial adhesion is even stronger than the hydrogel cohesion, with the hydrogel itself breaking before it peels off of the surface.

Currently, both the synthesized thin hydrogel coating and the bulk films are mechanically strong enough to maintain their structural integrity during the tribological testing process (described below). The thickness of the coated hydrogel films is at the range of 10˜500 We are in the process to load various prophylactic drugs into the hydrogels and test drug releasing profiles as a function of friction. For example, Tenofovir has demonstrated therapeutic effects against HIV transmission, and its hydrophilic structure helps its encapsulation into the hydrogel. The drug release profile can be affected by the applied shear force, pH values, and by having different hydrogel compositions, enabling optimization of the final design of drug-loaded hydrogels.

Mechanical and Tribological Testing of Bulk and Thin Coating Hydrogels:

Macro- and micro-scale mechanical testing of the synthesized hydrogels was performed to characterize their mechanical properties and lubricating capabilities and develop a deeper understanding of the lubricating mechanisms. Understanding both the macroscale performance and the fundamental mechanics enabling this performance facilitates the rational design of condoms and related applications that are intrinsically lubricating.

The modulus of the gel samples was tested with standard mechanical tensile tests. Dogbone samples were prepared with a cross sectional area of 136 mm×3.175 mm. Once loaded into the tensile testing machine (Series IX-5500, MTS) the samples were pulled, using displacement control, to failure at a crosshead speed of 2.5 mm/min. Load data was collected using a 10N sensitivity load cell and displacement data was collected with an extensometer. Load vs. displacement curves where translated into stress vs. strain curves using

$$σ = \frac{F}{A} and ϵ = \frac{l - lo}{lo}$$

respectively. A line was fit to the stress strain curves which determined the elastic modulus for the 254 nm gel sample to be 9.8±0.69 MPa.

For physiologically-relevant assessment of intrinsically lubricating hydrogels as condom materials, macro-scale testing is conducted using a mini-traction machine (MTM) focused on physiological compressive pressures, sliding speeds, and temperatures. In an aqueous environment (at room temperature and at body temperatures, e.g. 22° C. and 37-39° C.) with a soft material as the counter-surface (e.g. natural rubber o-ring), the frictional properties of bulk hydrogels synthesized with different crosslinker densities were measured. The friction behavior is reported as the traction coefficient, defined as the lateral force divided by the applied force, equivalent to the friction coefficient. The applied force yielded contact pressures estimated to be in the appropriate range of biological contact pressures (e.g. 50-350 kPa). At a constant speed of 100 mm/s we observed a decrease in friction coefficient for bulk hydrogels with lower crosslinker densities. There was a trade-off, though, where the lower crosslinker hydrogels also exhibited increased deformation/wear of the sample.

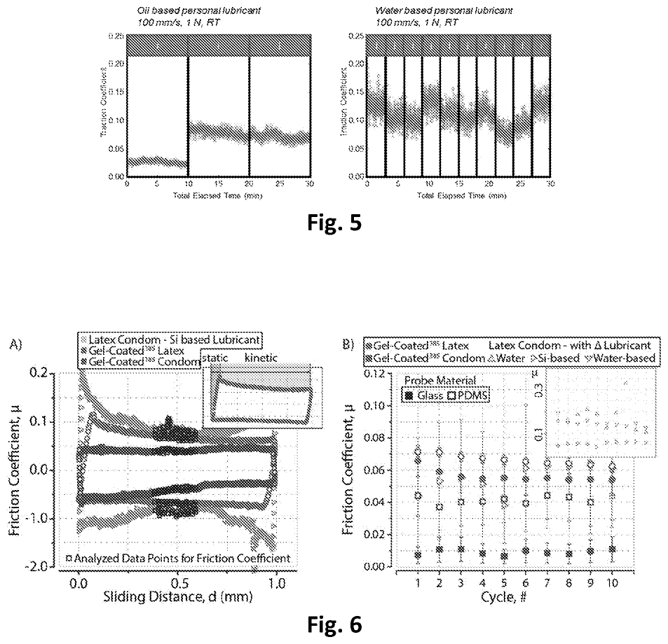

When hydrogels were synthesized as a thin coating (approximately 150 μm thick) instead of as a bulk material, under the same MTM testing conditions (39° C., 1 N, 100 mm/s sliding speed) the coatings maintained their intrinsically low friction coefficient. Moreover, withstanding up to one hour of sliding (FIG. 4, right plot), the hydrogel coating (friction coefficient: 0.07±0.02 averaged over one hour of testing) resulted in a 27-fold reduction in friction compared to a latex-on-latex contact (friction coefficient: 1.90±0.91, averaged over 1 minute of testing, after which the test was terminated due to sample damage and to avoid risk of damaging the MTM). The friction coefficient values were also comparable to and often lower than values obtained for latex sliding on latex with no hydrogel coating, but using commercial personal lubricants, either oil-based (FIG. 5, left) or water based (FIG. 5, right). Repeated tests (at room temperature, ca. 22° C.) are shown under the same conditions as tests marked “i” in FIG. 4. The water-based lubricant overall exhibited higher friction coefficient than the coated latex: 0.11±0.02 averaged over 30 minutes vs. 0.07±0.02 averaged over 60 minutes. For a direct comparison, hydrogel coated latex was also measured at room temperature (22° C., 1 N, 100 mm/s), with a friction coefficient of 0.05±0.02 and 0.06±0.02 averaged over two separate 5 minute increments. The oil-based lubricant overall exhibited a friction coefficient nominally the same as the coated latex: 0.06±0.02 averaged over 30 minutes, vs. hydrogel coating values of 0.07±0.02 averaged over 60 minutes at 39° C., or 0.05-0.06±0.02 averaged over 2 5-minute periods at 22° C. However, note that oil-based lubricants cannot be used in practice with latex condoms because the oil will dissolve the condom, leading to breakage. The test here is included to show that the hydrogel-coated latex reaches nearly the same lubrication performance as an oil-based lubricant, but without introducing the increased probability of breakage. Additional sliding speeds and contact pressures in the macroscale contact were also examined (specified in FIG. 4), with the coatings maintaining low friction coefficient for these broader conditions.

Micro-scale tribological testing was used to guide the material synthesis and supplement the macro-scale testing through examination of hydrogel sliding and contact mechanics. A micro indentation and tribology instrument has been developed at UPenn to test micro- and meso-scale properties of hydrogels. This instrument can apply contact pressures and sliding speeds much lower than the MTM (<1 kPa, 100 μm/s). At these pressures and speeds, molecular dynamics begins to influence the mechano-tribological behavior of the hydrogels more than the fluid dynamics governing more severe conditions. Understanding these fundamental mechanics will help direct synthesis of both bulk and thin film gels as well as be a signal for further characterization with the MTM.

Micro-scale friction experiments showed uniform low friction for all uncoated latex condom samples with personal lubricant, for gel-coated latex condom samples, and for gel-coated latex sheets, all of which were tested against a glass and a PDMS slider (FIG. 6). The friction loops for characteristic experiments are shown in FIG. 6a. Gel-coated samples displayed a low static coefficient of friction regardless of the slider material, (FIG. 6b) and remained consistent for several cycles of testing. This suggests that discomfort due to initial, unlubricated sliding or reversal of direction will be low. Uncoated-latex condom samples, however, displayed a large static coefficient of friction which transitioned to a somewhat lower kinetic coefficient of friction as seen in FIG. 6a, at the left and right sides of the friction traces, but with kinetic friction coefficient values still larger than those found for the hydrogel-coated materials, even though the uncoated latex was tested in various commercial lubricants.

The Effect of Hydrogel Composition on Friction

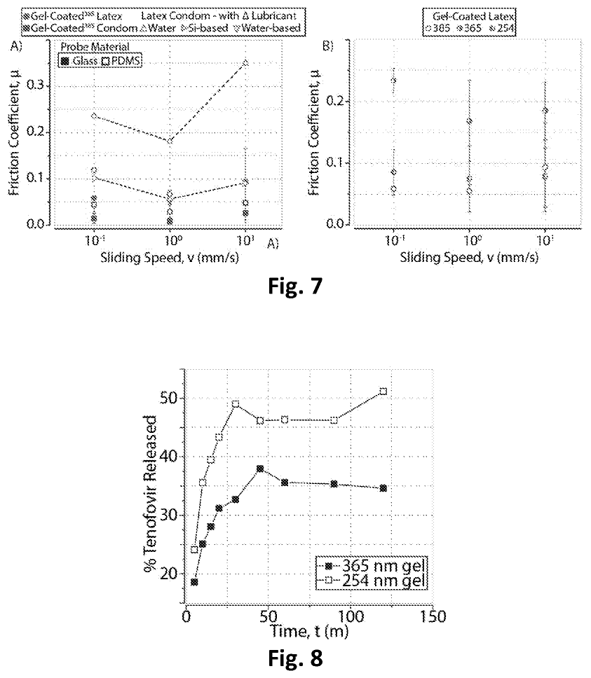

The effect of sliding speed and gel synthesis method on the friction coefficient is shown in FIG. 7. FIG. 7a shows the gel-coated condom performed the best out of all samples, for both probes, at all speeds. FIG. 7b shows the friction dependence on the UV light used to cure and graft the gels. The gel exposed to 385 nm-low intensity UV light had the lowest friction against a glass slider and the gel exposed to 254 nm-high intensity and 365 nm-high intensity UV light had higher friction. The intensity of the UV light causes the gel to be highly crosslinked and brittle at the surface, which corresponds to smaller mesh size and a higher friction coefficient.

Hydrogel Coatings on all Surfaces

While the method for developing hydrogel coatings on polymer surfaces produces strong surface binding, it is limited in the context of a broader array of applications since hydrophobic benzophenone cannot diffuse into inorganic materials (e.g. metals, glass). To overcome this, here we take advantage of diazonium chemistry to achieve surface attachment through covalent bonding, acting as an ideal “primer” layer for subsequent hydrogel attachment. Treatment of the primer layer forms reactive acrylate functional group as an anchoring point for the hydrogel (see example FTIR spectra). This produced tight surface binding (as demonstrated on a condom and PDMS surface)—with stretching, the hydrogel breaks before detaching from the condom). By eliminating the need for surface diffusion of the priming molecules, this method can be readily applied to non-polymer based surfaces.

1. A personal wellness product, comprising: a self-lubricating, tough hydrogel material, the hydrogel material optionally comprising a double interpenetrating network (D-IPN) matrix.

2. The personal wellness product of claim 1, the personal healthy product being characterized as a condom, a sexual health device, or a sexual pleasure device.

3. The personal wellness product of claim 1, wherein the personal wellness product is substantially free of any additional external lubricant.

4. The personal wellness product of claim 1, wherein the hydrogel material is disposed as a coating on a base material.

5. The personal wellness product of claim 4, wherein the base material comprises a polymer network, preferably elastomers, such as latex, polyurethane and silicone.

6. The personal wellness product of claim 1, wherein the hydrogel material is free-standing without a base material.

7. The personal wellness product of claim 1, wherein the hydrogel material comprises one or more medicaments.

8. The personal wellness product of claim 1, wherein the hydrogel material comprises one or more antifouling products.

9. The personal wellness product of claim 1, wherein the hydrogel material comprises one or more of chitosan, hyaluronic acid (HA), alginate, polyacrylamide (PAm), poly(acrylic acid) (PAA), poly(methacrylic acid) (PMAA), poly(vinyl alcohol) (PVA), and poly({2-[methacryloyloxy]ethyl}trimethylammonium chloride) (PMETAC).

10. The personal wellness product of claim 1, wherein the hydrogel comprises from 75-90 wt % water and from 10-25 wt % combined of acrylamide and alginate.

11. The personal wellness product of claim 1, wherein the product is characterized as having a friction coefficient in the range of from about 1 or less.

12. A medical device, comprising: self-lubricating hydrogel materials.

13. The medical device of claim 12, wherein the medical device is characterized as a contact lens, a hygiene product, a tissue engineering scaffold, a drug delivery carrier, a wound dressing, a needle, a catheter, a cannula, a trocar, an endotracheal tube, an endoscope, a cutting edge, a valve, and stopcocks.

14. The medical device of claim 12, wherein the hydrogel is disposed as a coating on a base material.

15. The medical device of claim 12, wherein the hydrogel material is free-standing without a base material.

16. The medical device of claim 12, wherein the hydrogel material comprises one or more medicaments.

17. The medical device of claim 12, wherein the hydrogel material comprises one or more antifouling products.

18. The medical device of claim 12, wherein said hydrogel comprises one or more of chitosan, hyaluronic acid (HA), alginate, polyacrylamide (PAm), poly(acrylic acid) (PAA), poly(methacrylic acid) (PMAA), poly(vinyl alcohol) (PVA), and poly({2-[methacryloyloxy]ethyl}trimethylammonium chloride) (PMETAC).

19. The medical device of claim 12, wherein the hydrogel comprises from 75-90 wt % water and from 10-25 wt % combined of acrylamide and alginate.

20. The medical device of claim 12, wherein the product is characterized as having a friction coefficient is 1 or lower.

21. A method of forming a medical product, comprising:
treating a primer layer so as to form one or more reactive acrylate functional groups or initiator configured to serve as anchoring points for a hydrogel material and
anchoring the hydrogel material to the primer layer.

22. The method of claim 21, wherein said hydrogel material comprises one or more of biocompatible and bioactive polymers such as chitosan, hyaluronic acid (HA), alginate, polyacrylamide (PAm), poly(acrylic acid) (PAA), poly(methacrylic acid) (PMAA), poly(vinyl alcohol) (PVA), and poly({2-[methacryloyloxy]ethyl}trimethylammonium chloride) (PMETAC).

23. The method of claim 21, wherein the hydrogel material is characterized as a double network material.
